(19) United States
(12) Patent Application Publication
(10) Pub. No.: US 2026/0124605 A1
(43) Pub. Date: May 7, 2026

(54) CATALYSTS, SYSTEMS, AND METHODS FOR METHANE PRODUCTION

(71) Applicant: Khalifa University of Science and Technology, Abu Dhabi (AE)

(72) Inventors: Kyriaki POLYCHRONOPOULOU, Abu Dhabi (AE); Yarjan Abdul SAMAD, Abu Dhabi (AE); Fahad ALABSI, Abu Dhabi (AE); Aseel Gamal Suliman HUSSIEN, Abu Dhabi (AE); Rami Adham Sayed Hassan ELKAFFAS, Abu Dhabi (AE)

(21) Appl. No.: 19/357,721

(22) Filed: Oct. 14, 2025

Related U.S. Application Data
(60) Provisional application No. 63/714,998, filed on Nov. 1, 2024.
(60) Provisional application No. 63/793,321, filed on Apr. 23, 2025.

(51) Int. Cl. B01J 23/83; B01J 23/10; B01J 27/02; B01J 35/30; B01J 35/61; B01J 35/63; B01J 35/64; B01J 37/02; C07C 1/12

(52) U.S. Cl. CPC B01J 23/83; B01J 23/10; B01J 27/02; B01J 35/394; B01J 35/613; B01J 35/615; B01J 35/633; B01J 35/635; B01J 35/647; B01J 37/0201; C07C 1/12; C07C 2523/755

(57) ABSTRACT

A catalyst includes a catalytically active material; and a support including: a carbon-containing material including at least one of graphene and graphite, wherein the carbon-containing material includes less than 5 wt. % oxygen based on elemental analysis of the carbon-containing material; and at least one of cerium oxide and praseodymium oxide.

### CROSS-REFERENCE TO RELATED APPLICATIONS

This application claims the benefit of and priority to U.S. provisional application 63/714,998, titled “GRAPHENE COMPOSITES FOR DECARBONIZATION”, filed Nov. 1, 2024, and U.S. provisional application 63/793,321, titled “GRAPHENE COMPOSITES FOR DECARBONIZATION”, filed Apr. 23, 2025, the contents of the aforementioned are incorporated by reference herein.

### TECHNICAL FIELD

The subject matter disclosed herein relates to the fields of chemical processing and energy conversion and more particularly to catalysts for catalytic chemical reactions. The present disclosure further includes methods for forming such catalysts.

### BACKGROUND

CO₂ emissions is one of the leading causes of global warming. Converting CO₂ into more value chemicals such as acetic acid, acetaldehyde, and methane is a way to combat the catastrophic effects of global warming. Carbon dioxide (CO₂) methanation (Eq. 1) offers an alternative approach to the reduction of CO₂ emissions by chemically hydrogenating CO₂ to give CH₄, which has higher volumetric energy content and safety insurance than H₂. The reaction for carbon dioxide methanation follows:

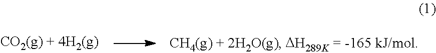

Various noble metal-based catalysts (Rh, Pd, and Ru) promote carbon dioxide methanation under mild conditions. However, the high cost of noble metals make these catalysts economically infeasible for industrialization. On the other hand, transition metals such as Ni, Fe, and Co are more economically feasible than noble metal counterparts, but these transition metals tend to deactivate due to carbon formation and sintering at high temperatures. Hence, tailoring a catalyst with enhanced activity and stability toward methanation is important for the industrialization of this process.

### SUMMARY

According to one aspect, a catalyst includes a catalytically active material; and a support including: a carbon-containing material including at least one of graphene and graphite, wherein the carbon-containing material includes less than 5 wt. % oxygen based on elemental analysis of the carbon-containing material; and at least one of cerium oxide and praseodymium oxide.

According to another aspect, a catalyst support includes graphite including less than 5 wt. % oxygen based on elemental analysis of the graphite; and a cerium praseodymium mixed oxide, wherein a weight ratio of cerium to praseodymium ranges from about 3:1 to about 5:1.

According to another aspect, a process for methanation includes introducing a feed stream to a catalyst at a process temperature sufficient to form methane, wherein the feed stream includes hydrogen; wherein the catalyst includes nickel and a support including (1) at least one of graphene and graphite; and (2) at least one of cerium oxide and praseodymium oxide.

### BRIEF DESCRIPTION OF THE DRAWINGS

FIG. 1 illustrates a method for methanation, according to some embodiments.

FIG. 2 illustrates x-ray diffraction (XRD) patterns of various catalysts and graphene, according to some embodiments.

FIG. 3 illustrates Raman spectra of various catalyst materials and graphene, according to some embodiments.

FIG. 4A illustrates deconvoluted Raman spectra of a Ni/CePr/GR3 catalyst, according to some embodiments.

FIG. 4B illustrates deconvoluted Raman spectra of a Ni/CePr/GR3 catalyst after carbon dioxide methanation, according to some embodiments.

FIG. 5 illustrates a bar chart depicting carbon dioxide conversion of various catalysts during carbon dioxide methanation, according to some embodiments.

FIG. 6A illustrates stability testing (carbon dioxide conversion) for a Ni/GR3 catalyst and a Ni/CePr/GR3 catalyst, according to some embodiments.

FIG. 6B illustrates stability testing (methane yield) for a Ni/GR3 catalyst and a Ni/CePr/GR3 catalyst, according to some embodiments.

FIG. 6C illustrates stability testing (methane selectivity) for a Ni/GR3 catalyst and a Ni/CePr/GR3 catalyst, according to some embodiments.

FIG. 6D illustrates stability testing (carbon monoxide selectivity) for a Ni/GR3 catalyst and a Ni/CePr/GR3 catalyst, according to some embodiments.

FIG. 6E illustrates stability testing (carbon monoxide yield) for a Ni/GR3 catalyst and a Ni/CePr/GR3 catalyst, according to some embodiments.

FIG. 7A illustrates stability testing (carbon dioxide conversion) for a Ni/CePr/GRP catalyst, according to some embodiments.

FIG. 7B illustrates stability testing (methane yield) for a Ni/CePr/GRP catalyst, according to some embodiments.

FIG. 7C illustrates stability testing (carbon monoxide selectivity) for a Ni/CePr/GRP catalyst, according to some embodiments.

FIG. 7D illustrates stability testing (methane selectivity) for a Ni/CePr/GRP catalyst, according to some embodiments.

FIG. 8 illustrates a comparative example of carbon dioxide conversion of various catalysts, according to some embodiments.

FIG. 9A illustrates the effect of temperature and space velocity on carbon dioxide methanation performance (carbon dioxide conversion) for the Ni/CePr/GR3 catalyst, according to some embodiments.

FIG. 9B illustrates the effect of temperature and space velocity on carbon dioxide methanation performance (carbon monoxide yield) for the Ni/CePr/GR3 catalyst, according to some embodiments.

FIG. 9C illustrates the effect of temperature and space velocity on carbon dioxide methanation performance (methane yield) for the Ni/CePr/GR3 catalyst, according to some embodiments.

FIG. 10A illustrates the effect of time on stream on the carbon dioxide methanation performance (carbon dioxide conversion) of the Ni/CePr/GR3 catalyst, according to some embodiments.

FIG. 10B illustrates the effect of time on stream on the carbon dioxide methanation performance (carbon monoxide yield) of the Ni/CePr/GR3 catalyst, according to some embodiments.

FIG. 10C illustrates the effect of time on stream on the carbon dioxide methanation performance (methane yield) of the Ni/CePr/GR3 catalyst, according to some embodiments.

FIG. 11 illustrates sulfur addition to the Ni/CePr/GR3 catalyst, according to some embodiments.

FIG. 12 illustrates x-ray diffraction (XRD) patterns of a Ni/CePr/GR3 and a Ni/CePr/GR3/S catalyst with 1 wt. % sulfur, according to some embodiments.

FIG. 13A illustrates performance (carbon dioxide conversion) of the Ni-supported catalysts (Ni/CePr/GR3 and Ni/CePr/GR3/S) as a function of reaction temperature, according to some embodiments.

FIG. 13B illustrates performance (methane yield) of the Ni-supported catalysts (Ni/CePr/GR3 and Ni/CePr/GR3/S) as a function of reaction temperature, according to some embodiments.

FIG. 13C illustrates performance (methane selectivity) of the Ni-supported catalysts (Ni/CePr/GR3 and Ni/CePr/GR3/S) as a function of reaction temperature, according to some embodiments.

FIG. 13D illustrates performance (carbon monoxide selectivity) of the Ni-supported catalysts (Ni/CePr/GR3 and Ni/CePr/GR3/S) as a function of reaction temperature, according to some embodiments.

FIG. 13E illustrates performance (carbon monoxide yield) of the Ni-supported catalysts (Ni/CePr/GR3 and Ni/CePr/GR3/S) as a function of reaction temperature, according to some embodiments.

FIG. 13F illustrates performance (magnified carbon monoxide yield) of the Ni-supported catalysts (Ni/CePr/GR3 and Ni/CePr/GR3/S) as a function of reaction temperature, according to some embodiments.

FIG. 14 illustrates x-ray diffraction (XRD) patterns for Ni/CePr/GT and Ni/GT, according to some embodiments.

FIG. 15A illustrates Raman spectra for Ni/CePr/GT and Ni/GT, according to some embodiments.

FIG. 15B illustrates deconvoluted Raman spectra for Ni/CePr/GT and Ni/GT, according to some embodiments.

FIG. 16A illustrates N₂ adsorption-desorption isotherms for Ni/CePr/GT and Ni/GT, according to some embodiments.

FIG. 16B illustrates the cumulative pore volume for Ni/CePr/GT and Ni/GT, according to some embodiments.

FIG. 17A illustrates performance (carbon dioxide conversion) of the Ni-supported catalysts (Ni/GR3, Ni/CePr/GR3, Ni/GT, and Ni/CePr/GT) as a function of reaction temperature, according to some embodiments.

FIG. 17B illustrates performance (methane yield) of the Ni-supported catalysts (Ni/GR3, Ni/CePr/GR3, Ni/GT, and Ni/CePr/GT) as a function of reaction temperature, according to some embodiments.

FIG. 17C illustrates performance (methane selectivity) of the Ni-supported catalysts (Ni/GR3, Ni/CePr/GR3, Ni/GT, and Ni/CePr/GT) as a function of reaction temperature, according to some embodiments.

FIG. 17D illustrates performance (carbon monoxide selectivity) of the Ni-supported catalysts (Ni/GR3, Ni/CePr/GR3, Ni/GT, and Ni/CePr/GT) as a function of reaction temperature, according to some embodiments.

FIG. 17E illustrates performance (carbon monoxide yield) of the Ni-supported catalysts (Ni/GR3, Ni/CePr/GR3, Ni/GT, and Ni/CePr/GT) as a function of reaction temperature, according to some embodiments.

FIG. 17F illustrates performance (magnified carbon monoxide yield) of the Ni-supported catalysts (Ni/GR3, Ni/CePr/GR3, Ni/GT, and Ni/CePr/GT) as a function of reaction temperature, according to some embodiments.

FIG. 18A illustrates stability testing (carbon dioxide conversion) of the Ni-supported catalysts (Ni/GR3, Ni/CePr/GR3, and Ni/CePr/GT) as a function of reaction temperature, according to some embodiments.

FIG. 18B illustrates stability testing (methane yield) of the Ni-supported catalysts (Ni/GR3, Ni/CePr/GR3, and Ni/CePr/GT) as a function of reaction temperature, according to some embodiments.

FIG. 18C illustrates stability testing (methane selectivity) of the Ni-supported catalysts (Ni/GR3, Ni/CePr/GR3, and Ni/CePr/GT) as a function of reaction temperature, according to some embodiments.

FIG. 18D illustrates stability testing (carbon monoxide selectivity) of the Ni-supported catalysts (Ni/GR3, Ni/CePr/GR3, and Ni/CePr/GT) as a function of reaction temperature, according to some embodiments.

FIG. 18E illustrates stability testing (carbon monoxide yield) of the Ni-supported catalysts (Ni/GR3, Ni/CePr/GR3, and Ni/CePr/GT) as a function of reaction temperature, according to some embodiments.

FIG. 18F illustrates stability testing (carbon monoxide yield) of the Ni-supported catalysts (Ni/GR3, Ni/CePr/GR3, and Ni/CePr/GT) as a function of reaction temperature, according to some embodiments.

FIG. 19 illustrates a bar chart depicting carbon dioxide conversion of various catalysts during carbon dioxide methanation, according to some embodiments.

### DETAILED DESCRIPTION

Embodiments of the present disclosure provide catalysts and support materials thereof. Catalysts of the present disclosure can efficiently promote the conversion of carbon dioxide and/or carbon monoxide in a methanation reaction. The methanation reaction is a chemical process where carbon monoxide (CO) and/or carbon dioxide (CO₂) are converted into methane (CH₄) and water (H₂O), using hydrogen (H₂). One example methanation reaction is the Sabatier reaction, which uses carbon dioxide as a feedstock. Various noble metal-based catalysts (Rh, Pd, and Ru) have conventionally been utilized. However, the high cost of noble metals makes these catalysts economically infeasible for industrialization. On the other hand, transition metals such as Ni, Fe, and Co are more economically feasible than noble metal counterparts, but they conventionally tend to deactivate due to carbon formation and sintering at high temperatures. Hence, tailoring a catalyst with enhanced activity and stability toward methanation is important for the industrialization of this process.

As used herein, the terms “catalyst”, “catalytic material”, or the like can refer to material which enables a chemical reaction to proceed at a faster rate or under different conditions (e.g., at a lower temperature) than otherwise possible, or to control a chemical reaction to generate particularly desired products. The catalysts of the present disclosure may include mixtures of two or more catalytic material(s) with other inert materials. The catalytic materials used in the present disclosure may be formed into desired shapes or sizes. The catalytic materials of the present disclosure can be pre-reduced catalyst precursors.

The catalyst includes a catalytically active material and a support. The catalytically active material at least partially facilitates the chemical reaction and provides the active sites where reactants are adsorbed, transformed, and/or desorbed. The catalytically active material can include one or more catalytically active metals capable of promoting the methanation reaction (e.g., carbon dioxide methanation and/or carbon monoxide methanation). Carbon dioxide methanation, which may be referred to as the Sabatier reaction, offers an alternative approach to the reduction of CO₂ emissions by chemically hydrogenating CO₂ to give CH₄, which has higher volumetric energy content and safety insurance than H₂. The reaction for carbon dioxide methanation follows:

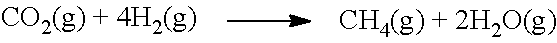

Carbon monoxide methanation is a catalytic process that converts carbon monoxide (CO) and hydrogen (H₂) into methane (CH₄) and water (H₂O). The reaction for carbon monoxide methanation follows:

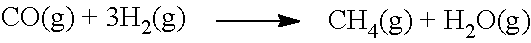

In one example, the catalytically active material includes at least one of nickel, ruthenium, platinum, palladium, and rhodium. In one non-limiting example, the catalytically active material includes nickel.

The weight percentage of the catalytically active material can be tuned to promote desirable results, such as conversion and/or selectivity. In one example, the weight percentage of the catalytically active material in the catalyst ranges from about 5 wt. % to about 60 wt. %. In another example, the weight percentage of the catalytically active material in the catalyst ranges from about 10 wt. % to about 50 wt. %. In another example, the weight percentage of the catalytically active material in the catalyst ranges from about 7 wt. % to about 40 wt. %. In another example, the weight percentage of the catalytically active material in the catalyst ranges from about 5 wt. % to about 30 wt. %. The weight percentage of the catalytically active material in the catalyst can be greater than about 1 wt. %, greater than about 3 wt. %, greater than about 4 wt. %, greater than about 5 wt. %, greater than about 10 wt. %, or values therebetween.

The catalytically active material can be dispersed on/within a support. The support can improve dispersion of the catalytically active material and enhance the catalytic properties of the active metal. Surface area, pore structure, and the presence of specific sites (e.g., oxygen vacancies) on the support can be tuned to promote efficient performance during operation of the methanation reaction. The support can be formed into predetermined shapes. For example, the support can take the form of spherical particles or beads, porous beads, pellets, tubes, Raschig rings, Super Raschig rings, Pall rings, Bielecki rings, extrudates, lobes, and/or saddles.

The support includes one or more carbon-containing materials. The weight percentage of carbon in the carbon-containing material is generally greater than 80 wt. %. In one example, the weight percentage of carbon in the carbon-containing material can be greater than 90 wt. %. In another example, the weight percentage of carbon in the carbon-containing material can be greater than 95 wt. %. In another example, the weight percentage of carbon in the carbon-containing material can be greater than 98 wt. %. Weight percentages of carbon in the carbon-containing material can be determined by elemental analysis, such as EDS (Energy Dispersive X-ray Spectroscopy) analysis.

In one example, the carbon-containing material is substantially free of oxygen-containing functional groups. In one example, the weight percentage of oxygen in the carbon-containing material is less than 20 wt. %, based on elemental analysis. In another example, the weight percentage of oxygen in the carbon-containing material is less than 10 wt. %, based on elemental analysis. In another example, the weight percentage of oxygen in the carbon-containing material is less than 5 wt. %, based on elemental analysis. In another example, the weight percentage of oxygen in the carbon-containing material ranges from 0 wt. % to 10 wt. %, based on elemental analysis. In another example, the weight percentage of oxygen in the carbon-containing material ranges from 0.1 wt. % to 5 wt. %, based on elemental analysis. Weight percentages of oxygen in the carbon-containing material can be determined by EDS (Energy Dispersive X-ray Spectroscopy) analysis.

In one embodiment, the carbon-containing material includes graphene. Graphene includes a two-dimensional sheet including carbon atoms arranged as a hexagonal lattice. The graphene can include plasma-formed graphene. Plasma-formed graphene is formed by decomposing carbon-containing precursors in a high-temperature plasma, such as microwave plasma, to create a high-energy environment that rearranges carbon atoms into a single sheet of hexagonally bonded carbon (e.g., single-layer graphene). The carbon-containing precursors, such as ethanol or methane, can be introduced into a plasma-filled reactor, and the plasma breaks down the carbon-containing precursors into carbon atoms and other molecules. As discussed, the graphene includes a two-dimensional sheet including carbon atoms arranged as a hexagonal lattice (e.g., distinct from a carbon nanotube having a cylindrical structure).

Graphene is generally substantially free of oxygen-containing functional groups. In one example, the weight percentage of oxygen in graphene is less than 5 wt. %, based on elemental analysis. In another example, the weight percentage of oxygen in graphene is less than 3 wt. %, based on elemental analysis. In another example, the weight percentage of oxygen in graphene is less than 1 wt. %, based on elemental analysis. In another example, the weight percentage of oxygen in graphene ranges from 0 wt. % to 4 wt. %, based on elemental analysis. In another example, the weight percentage of oxygen in graphene ranges from 1 wt. % to 5 wt. %, based on elemental analysis. Weight percentages of oxygen in graphene can be determined by EDS (Energy Dispersive X-ray Spectroscopy) analysis.

In one non-limiting example, compared to reduced graphene oxide (rGO) that can include 10 wt. % to 30 wt. % oxygen based on elemental analysis (or greater for graphene oxide), graphene of the present disclosure includes fewer or no oxygen-containing functional groups. Reduced graphene oxide (rGO) is prepared by chemically or thermally reducing graphene oxide, and although it may partially restore sp² conjugation, reduced graphene oxide still contains residual oxygen-containing functional groups (e.g. hydroxyl, epoxy, carbonyl) and structural defects that can reduce stability. In one example, the support of the present disclosure is free of reduced graphene oxide (rGO). In another example, the support of the present disclosure is free of carbon nanofibers. In another example, the support of the present disclosure is not nitrogen doped.

A weight percentage of carbon in the graphene, such as plasma-formed graphene, can be greater than 93 wt. %. In one example, the weight percentage of carbon in the graphene, such as plasma-formed graphene, can be greater than 95 wt. %. In another example, the weight percentage of carbon in the graphene, such as plasma-formed graphene, can be greater than 97 wt. %. In another example, the weight percentage of carbon in the graphene, such as plasma-formed graphene, can be greater than 99 wt. %. In one non-limiting example, compared to a pyrolysis-formed graphene material having a weight percentage of carbon ranging from 80 wt. % to 93 wt. %, the weight percentage of carbon in plasma-formed graphene can be greater than 93 wt. %. In another non-limiting example, since the plasma-formed graphene includes less impurities compared to pyrolysis-formed graphene, the plasma-formed graphene exhibits higher conversions for methanation. For example, pyrolysis-formed graphene can further include iron and silicon, based on elemental analysis. In one example, pyrolysis-formed graphene includes greater than 0.1 wt. % iron, greater than 0.4 wt. % silicon, and greater than 0.3 wt. % sulfur. Weight percentages of carbon in the graphene can be determined by EDS (Energy Dispersive X-ray Spectroscopy) analysis.

Graphene used in the support of the present disclosure can exhibit a density greater than about 0.01 g/cm³. In one example, graphene exhibits a density greater than 0.05 g/cm³. In another example, graphene exhibits a density greater than 0.1 g/cm³. In another example, graphene exhibits a density greater than 1 g/cm³. In another example, graphene exhibits a density greater than 2 g/cm³. In another example, graphene exhibits a density ranging from 1 g/cm³ to 3 g/cm³. In one non-limiting example, compared to a graphene aerogel material that exhibits a density of about 0.00016 g/cm³, graphene of the present disclosure can exhibit a density greater than about 0.01 g/cm³, greater than about 0.1 g/cm³, or greater than 1 g/cm³.

In one example, when the support includes graphene, the support exhibits a BET surface area greater than about 90 m²/g. In another example, when the support includes graphene, the support exhibits a BET surface area ranging from about 80 m²/g to about 140 m²/g. In one example, when the support includes graphene, the support exhibits an average pore volume ranging from about 0.3 cm³/g to about 0.6 cm³/g. In one example, when the support includes graphene, the support exhibits an average pore size ranging from about 10 nm to about 25 nm, or from about 8 nm to about 18 nm.

In one embodiment, the carbon-containing material includes graphite. Graphite includes two or more layers of graphene. The graphite of the present disclosure can include natural graphite and/or modified graphite. Modified graphite can be made from carbon-rich materials, such as through high-temperature processing. In one example, the modified graphite includes laser-expanded graphite. Laser-expanded graphite is graphite that has undergone expansion using laser technology to create a porous structure with enhanced properties. In one example, graphite of the present disclosure includes at least 20 layers of graphene. In another example, graphite of the present disclosure includes at least 50 layers of graphene. In another example, graphite of the present disclosure includes at least 100 layers of graphene.

Graphite is generally substantially free of oxygen-containing functional groups. In one example, the weight percentage of oxygen in graphite is less than 5 wt. %, based on elemental analysis. In another example, the weight percentage of oxygen in graphite is less than 3 wt. %, based on elemental analysis. In another example, the weight percentage of oxygen in graphite is less than 1 wt. %, based on elemental analysis. In another example, the weight percentage of oxygen in graphite ranges from 0 wt. % to 4 wt. %, based on elemental analysis. In another example, the weight percentage of oxygen in graphite ranges from 1 wt. % to 5 wt. %, based on elemental analysis. In one non-limiting example, compared to reduced graphene oxide (rGO) that can include 10 wt. % to 30 wt. % oxygen based on elemental analysis (or greater for graphene oxide), graphite of the present disclosure includes less or no oxygen-containing functional groups. Weight percentages of oxygen in graphite can be determined by EDS (Energy Dispersive X-ray Spectroscopy) analysis.

A weight percentage of carbon in the graphite, such as laser-expanded graphite, can be greater than 93 wt. %. In one example, the weight percentage of carbon in the graphite, such as laser-expanded graphite, can be greater than 95 wt. %. In another example, the weight percentage of carbon in the graphite, such as laser-expanded graphite, can be greater than 97 wt. %. In another example, the weight percentage of carbon in the graphite, such as laser-expanded graphite, can be greater than 99 wt. %.

Graphite used in the support of the present disclosure can exhibit a density greater than about 0.001 g/cm³. In one example, graphite exhibits a density greater than 0.05 g/cm³. In another example, graphite exhibits a density greater than 0.1 g/cm³. In another example, graphite exhibits a density greater than 1 g/cm³. In another example, graphite exhibits a density greater than 2 g/cm³. In one example, laser-expanded graphite can exhibit a density of less than 1 g/cm³. In another example, laser-expanded graphite can exhibit a density of less than 0.1 g/cm³. Laser-expanded graphite can exhibit an expansion volume of greater than 50 mL/g, where the expansion volume is a measure of the final volume in milliliters of the expanded material per gram of the original graphite. In one example, laser-expanded graphite exhibits an expansion volume of greater than 100 mL/g. In another example, laser-expanded graphite exhibits an expansion volume of greater than 200 mL/g.

In one example, when the support includes graphite, the support exhibits a BET surface area greater than about 60 m²/g. In another example, when the support includes graphite, the support exhibits a BET surface area ranging from about 40 m²/g to about 90 m²/g. In one example, when the support includes graphite, the support exhibits an average pore volume ranging from about 0.1 cm³/g to about 0.3 cm³/g. In one example, when the support includes graphite, the support exhibits an average pore size ranging from about 10 nm to about 20 nm, or from about 8 nm to about 15 nm.

The support can include cerium oxide (CeO₂). Cerium oxide exhibits oxygen storage capacity (OSC) and a redox property. Therefore, its presence can enhance the performance and coke resistance of the catalyst. Ceria can also act as a structural and electronic promoter, which improves the metal-support interaction. Furthermore, the oxygen vacancies in ceria lattice increase the rate of the reaction by adsorbing and activating a C—O bond. The support can include praseodymium oxide. The addition of praseodymium oxide can improve the coke resistance by increasing the basicity of the cerium oxide. This can be due at least in part by changes its intrinsic properties such as electronic configuration, coordination, oxidation state, and cation size.

A weight percentage of cerium oxide in the catalyst can range from about 0.01 wt. % to about 25 wt. %. In one example, a weight percentage of cerium oxide in the catalyst can range from about 1 wt. % to about 30 wt. %. In another example, a weight percentage of cerium oxide in the catalyst can range from about 10 wt. % to about 25 wt. %. In another example, a weight percentage of cerium oxide in the catalyst can range from about 12 wt. % to about 20 wt. %. In one non-limiting example, a weight percentage of cerium oxide in the catalyst can range from about 14 wt. % to about 18 wt. %. The weight percentage of cerium oxide in the catalyst can be about 14 wt. %, about 15 wt. %, about 16 wt. %, about 17 wt. %, about 18 wt. %, or values or ranges therebetween.

The support can include praseodymium oxide. A weight percentage of praseodymium oxide in the catalyst can range from about 0.01 wt. % to about 15 wt. %. In one example, a weight percentage of praseodymium oxide in the catalyst can range from about 1 wt. % to about 10 wt. %. In another example, a weight percentage of praseodymium oxide in the catalyst can range from about 2 wt. % to about 8 wt. %. In another example, a weight percentage of praseodymium oxide in the catalyst can range from about 0.1 wt. % to about 8 wt. %. In one non-limiting example, a weight percentage of praseodymium oxide in the catalyst can range from about 2 wt. % to about 6 wt. %. The weight percentage of praseodymium oxide in the catalyst can be about 2 wt. %, about 3 wt. %, about 4 wt. %, about 5 wt. %, about 6 wt. %, or values or ranges therebetween.

The support can include both cerium oxide and praseodymium oxide (e.g., in the form of a cerium praseodymium mixed oxide following the formula CeₓPr₁₋ₓO_{2-δ}). The addition of cerium oxide and praseodymium oxide can lead to an increase in specific surface area. Further, the addition of cerium oxide and praseodymium oxide act as stabilizers that prevent the agglomeration and restacking of graphene sheets. In one example, doping ceria with trivalent Pr⁺³ can enhance coke resistance by increasing ceria's basicity. Additionally, or alternatively, the support can include samarium, lanthanum, and/or oxides thereof. The samarium, lanthanum, and/or oxides thereof can be utilized according to one or more weight percentages discussed for praseodymium oxide of the present disclosure. For example, this can be attributed to changes in electronic configuration, coordination, oxidation state, and cation size.

The weight ratio of cerium to praseodymium can range from about 0.5:1 to about 12:1, based on elemental analysis. In one example, the weight ratio of cerium to praseodymium ranges from about 2:1 to about 6:1, based on elemental analysis. In another example, the weight ratio of cerium to praseodymium ranges from about 3:1 to about 6:1, based on elemental analysis. In another example, the weight ratio of cerium to praseodymium ranges from about 3:1 to about 5:1, based on elemental analysis. In another example, the weight ratio of cerium to praseodymium is about 4:1, based on elemental analysis. Elemental analysis can be performed using EDS (Energy Dispersive X-ray Spectroscopy) analysis.

A weight percentage of cerium oxide and praseodymium oxide in the catalyst can range from about 0.01 wt. % to about 25 wt. %. In one example, a weight percentage of cerium oxide and praseodymium oxide in the catalyst can range from about 1 wt. % to about 30 wt. %. In another example, a weight percentage of cerium oxide and praseodymium oxide in the catalyst can range from about 10 wt. % to about 22 wt. %. In another example, a weight percentage of cerium oxide and praseodymium oxide in the catalyst can range from about 15 wt. % to about 25 wt. %. In one non-limiting example, a weight percentage of cerium oxide and praseodymium oxide in the catalyst can range from about 17 wt. % to about 22 wt. %.

The support can exhibit an average pore size ranging from about 8 nm to about 30 nm. In one example, the support exhibits an average pore size ranging from about 10 nm to about 20 nm. In one example, the support exhibits an average pore size ranging from about 8 nm to about 22 nm. The catalyst can exhibit an average pore size ranging from about 8 nm to about 30 nm. In one example, the catalyst exhibits an average pore size ranging from about 8 nm to about 16 nm. Average pore size can be determined using mercury intrusion porosimetry, where mercury is forced into pores under controlled pressure, and the applied pressure is related to pore size.

The support can exhibit a BET (Brunauer-Emmett-Teller) surface area of greater than about 70 m²/g. In one example, the support exhibits a BET surface area of greater than about 80 m²/g. In another example, the support exhibits a BET surface area ranging from about 80 m²/g to about 100 m²/g. The catalyst can exhibit a BET surface area of greater than about 60 m²/g. In one example, the catalyst exhibits a BET surface area of greater than about 70 m²/g. In another example, the catalyst exhibits a BET surface area of greater than about 100 m²/g. The BET surface area can be determined based on how much gas, such as nitrogen, is adsorbed onto a surface of the catalyst/support.

The ID/IG ratio (a parameter from Raman spectroscopy that quantifies the degree of structural disorder in carbon-based materials) is calculated by dividing the intensity of the D-band (representing disorder) by the intensity of the G-band (representing structural order). The ID/IG ratio can be used to quantify the disorder and defects among different graphene-based materials, which can change surface properties and adsorption capability. The higher the ID/IG ratios, the more disorder. In one example, the catalyst exhibits an ID/IG ratio greater than 0.1. In another example, the catalyst exhibits an ID/IG ratio greater than 0.2. In another example, the catalyst exhibits an ID/IG ratio greater than 0.3. In one non-limiting example, the catalyst including nickel, graphite, and cerium praseodymium oxide can exhibit an ID/IG ratio greater than 0.3.

In one embodiment, the catalyst support is formed by mixing a praseodymium-containing salt (e.g., Pr(NO₃)₃) with a cerium-containing salt (e.g., Ce(NO₃)₃) in a liquid, such as water. An agent, such as ammonia, can be added to promote precipitation. Graphene and/or graphite can be doped using the obtained cerium-containing and praseodymium-containing precipitation product. The supported nickel-based catalyst can be prepared by wet impregnation. The support can be added to an aqueous solution containing a nickel-containing salt (e.g., 10 wt % of Ni(NO₃)₂) while mixing. The mixture can then be dried. The catalyst can be obtained by calcination, such as by calcining at 550° C. for 4 hours in air.

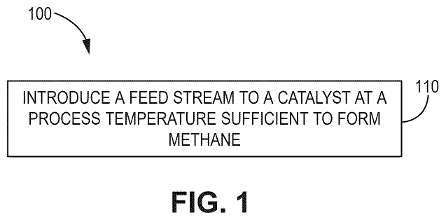

FIG. 1 illustrates a method for methanation, according to some embodiments. Method 100 includes one or more of the following aspects:

A feed stream is introduced 110 to a catalyst at a process temperature sufficient to form methane. Introducing 110 includes bringing one or more chemical species (reactant(s) in feed stream) into contact with the catalyst in such a way that they can interact (physically/chemically) and undergo a catalytic transformation to one or more products. The feed stream can include at least one of carbon dioxide, carbon monoxide, and hydrogen. In one embodiment, carbon dioxide and hydrogen are introduced as a single feed stream. In another embodiment, carbon dioxide and hydrogen are introduced as distinct feed streams. The feed stream can further include at least one of sulfur, one or more sulfur-containing compounds, one or more nitrogen-containing compounds, water, and carbon monoxide. Examples of sulfur-containing compounds include sulfur dioxide and sulfur trioxide. The catalyst includes a catalyst of the present disclosure.

Industrial gas feed streams can include various impurities, such as sulfur-containing compounds. Even very low levels of sulfur can greatly deactivate Ni catalysts by forming nickel sulfide phases, such as NiS and Ni₃S₂. In one example, one sulfur atom can poison ten or more Ni active sites due to its geometric blocking and electronic effects. The catalyst of the present disclosure efficiently promotes the methanation reaction, even in the presence of sulfur-containing compounds. In one example, one or more sulfur-containing compounds can be in contact with at least a portion of the catalyst and/or support during operation of the methanation reaction, and/or one or more sulfur-containing compounds can be placed in contact with at least a portion of the catalyst and/or support prior to performing the methanation reaction.

The process temperature can be a temperature of greater than 200° C. In one example, the process temperature is a temperature of greater than 250° C. In another example, the process temperature is a temperature of greater than 275° C. In another example, the process temperature is a temperature of greater than 300° C. In another example, the process temperature is a temperature of greater than 350° C. In another example, the process temperature is a temperature ranging from 225° C. to 400° C. In another example, the process temperature is a temperature ranging from 250° C. to 350° C.

The feed stream can be introduced 110 at a process pressure of about 1 bar. In one example, the feed stream can be introduced 110 at a process pressure ranging from about 1 bar to about 50 bar. In one example, the feed stream can be introduced 110 at a process pressure ranging from about 10 bar to about 30 bar. In another example, the feed stream can be introduced 110 at a process pressure ranging from about 5 bar to about 20 bar.

The feed stream can be introduced 110 at a weight hourly space velocity (the mass flow rate of the feed per hour divided by the weight of the catalyst in the reactor) of greater than 25,000 h⁻¹. The feed stream can be introduced 110 at a weight hourly space velocity ranging from 15,000 h⁻¹ to 100,000 h⁻¹. In one example, the feed stream is introduced 110 at a weight hourly space velocity ranging from 20,000 h⁻¹ to 75,000 h⁻¹. In another example, the feed stream is introduced 110 at a weight hourly space velocity ranging from 15,000 h⁻¹ to 40,000 h⁻¹. In another example, the feed stream is introduced 110 at a weight hourly space velocity ranging from 20,000 h⁻¹ to 30,000 h⁻¹.

Introducing 110 can be sufficient to form methane and water. Using the catalyst, a carbon dioxide conversion of greater than 60% can be achieved at the process temperature. In one example, a carbon dioxide conversion of greater than 70% can be achieved at the process temperature. In another example, a carbon dioxide conversion of greater than 80% can be achieved at the process temperature. In another example, a methane selectivity of greater than 90% can be achieved at the process temperature. In another example, a methane selectivity of greater than 95% can be achieved at the process temperature. In another example, a carbon monoxide selectivity of less than 3% can be achieved at the process temperature. In another example, a carbon monoxide selectivity of less than 2% can be achieved at the process temperature. Conversion, selectivity, and/or yield can be calculated using the process pressure and weight hourly space velocity of the present disclosure.

The catalyst can be positioned within a catalytic reactor for performing the methanation reaction. Accordingly, the catalyst of the present disclosure can efficiently promote the conversion of carbon dioxide in the methanation reaction. The catalyst of the present disclosure efficiently promotes the methanation reaction, even in the presence of sulfur-containing compounds. In some embodiments, the support includes at least one of graphene and graphite. The support can include cerium oxide and/or praseodymium oxide. The addition of praseodymium oxide can improve the coke resistance by increasing the basicity of cerium oxide.

### Example 1—Graphene-Containing Support

A catalyst may be referred to herein as “Ni/GR3”. “Ni” corresponds to nickel as an active metal, and “GR3” corresponds to plasma-formed graphene. The term “GRP” as used herein refers to pyrolysis-formed graphene. One example of a Ni/GR3 catalyst used for testing is a 10% Ni/GR3 catalyst.

A catalyst may be referred to herein as “Ni/CePr/GR3”. The term “CePr” refers to the inclusion of a cerium praseodymium mixed oxide following the formula CeₓPr₁₋ₓO_{2-δ}, where x represents cerium content and 6 represents oxygen vacancy. One example of a Ni/CePr/GR3 catalyst used for testing is a 10% Ni/20% (CePr)/GR3 catalyst, where the catalyst is formed using a weight ratio of cerium to praseodymium of 4:1.

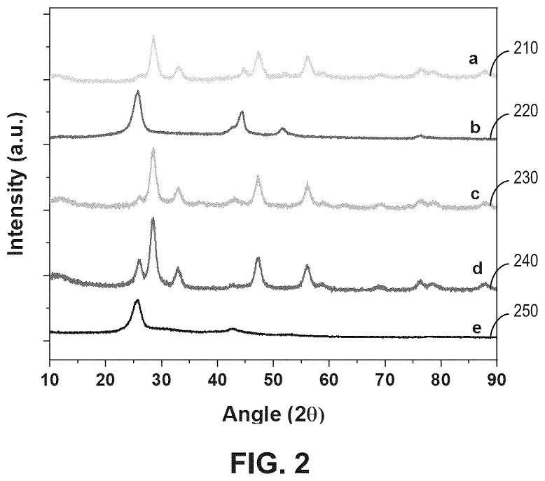

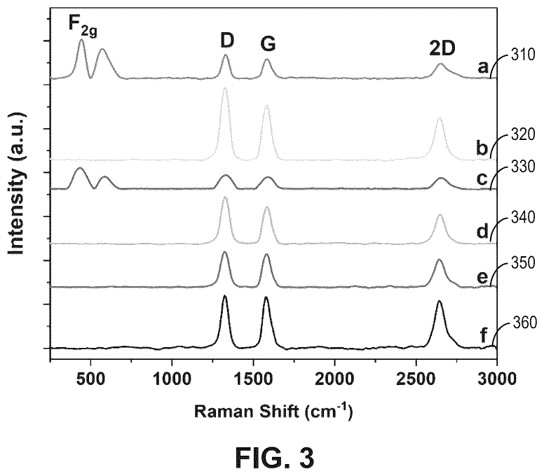

FIG. 2 illustrates x-ray diffraction (XRD) patterns of various catalysts and graphene, according to some embodiments. Reference numerals are as follows: (210) Ni/CePr/GR3, (220) Ni/GR3 after carbon dioxide methanation at 250° C., 300° C., and 350° C. for 1 h, (230) Ni/CePr/GR3, (240) Ni/GR3, and (250) Graphene Batch G2. FIG. 3 illustrates Raman spectra of various catalyst materials and graphene, according to some embodiments. Reference numerals are as follows: (310) Ni/CePr/GR3, (320) Ni/GR3 after carbon dioxide methanation at 250° C., 300° C., and 350° C. for 1 h, (330) Ni/CePr/GR3, (340) Ni/GR3, (350) Graphene Batch G2, and (360) Graphene Batch G1.

Preliminary characterization of the catalysts using X-ray Diffraction (XRD) and Raman Spectroscopy show that the formation of a cubic fluorite structure of ceria after the coprecipitation of Ce and Pr on the surface of graphene with the emergence of diffraction peaks at around 28.5, 33.1, 47.5 and 56° 20 correspond to reflections due to the (111), (200), (220) and (311) planes, respectively, of the cubic fluorite structure of CeO₂.

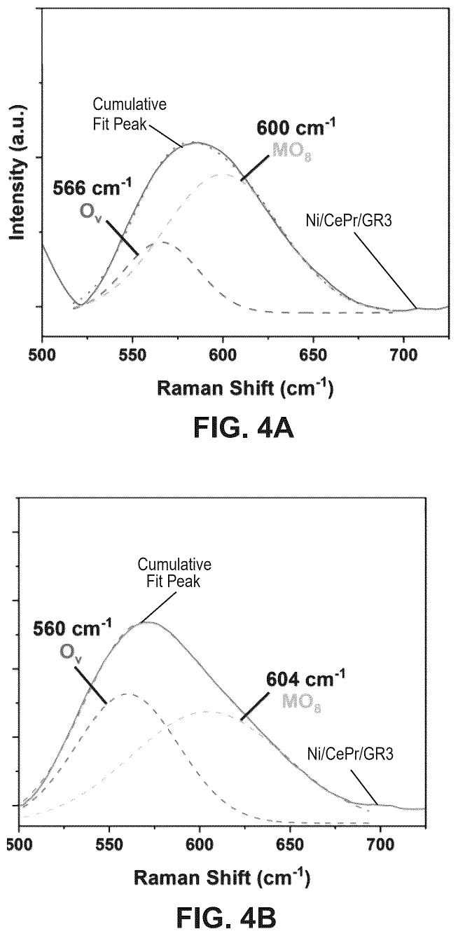

FIG. 4A illustrates deconvoluted Raman spectra of a Ni/CePr/GR3 catalyst, according to some embodiments. FIG. 4A shows the deconvoluted defective peak of 590 cm⁻¹, as this refers to the oxygen vacancies/defects in the lattice due to charge compensation. The deconvolution of the peak at 590 cm⁻¹ for catalysts was conducted to uncouple the formation of Oᵥ (coordinatively unsaturated site) and MOs-hetero-phase impurities contribution. FIG. 4B illustrates deconvoluted Raman spectra of a Ni/CePr/GR3 catalyst after carbon dioxide methanation, according to some embodiments. The deconvoluted Raman spectra is shown after carbon dioxide methanation at 250° C., 300° C., and 350° C. for 1 h.

The catalytic performance of the catalysts towards carbon dioxide methanation was tested using a continuous flow fixed-bed stainless steel reactor. 0.24 g of the fresh catalyst was loaded in the reactor and reduced for 3 hours using 50% H₂/He flow. Then, the catalytic activity was evaluated as a function of temperature (T=250° C., 300° C., 350° C.) with a total flow rate of 100 mL min⁻¹ (10 mL min⁻¹ CO₂, 40 mL min⁻¹ H₂ and 50 mL min⁻¹ He) and weight hourly space velocity (WHSV) of 25000 mL g_{cat}⁻¹ h⁻¹ and the temperature was maintained for 1 h. The effluent gases were continuously monitored using a GC-MS. For example, CO was detected as the only reaction by-product besides CH₄. CH4 selectivity, CO selectivity and CH₄ yield were calculated based on the following Eqs. (2)-(5). The stability tests (time-on-stream) were conducted for 100 h at 350° C. (chosen as high activity was achieved in this temperature) under 10 mL min⁻¹ CO, 40 mL min⁻¹ H₂ and 50 mL min⁻¹ He.

$${X}_{C {O}_{2}} ( \% ) = \frac{{C}_{C {H}_{4}}^{o u t} + {C}_{CO}^{o u t}}{{C}_{{CO}_{2}}^{o u t} + {C}_{C {H}_{4}}^{o u t} + {C}_{CO}^{o u t}} 100 \qquad ( 2 )$$

$${S}_{C {H}_{4}} ( \% ) = \frac{{C}_{C {H}_{4}}^{o u t}}{{C}_{C {H}_{4}}^{o u t} + {C}_{CO}^{o u t}} 100 \qquad ( 3 )$$

$${S}_{C O} ( \% ) = \frac{{C}_{CO}^{o u t}}{{C}_{C {H}_{4}}^{o u t} + {C}_{CO}^{o u t}} 100 \qquad ( 4 )$$

$${Y}_{C {H}_{4}} ( \% ) = \frac{{X}_{{CO}_{2}} \cdot {S}_{C {H}_{4}}}{100} \qquad ( 5 )$$

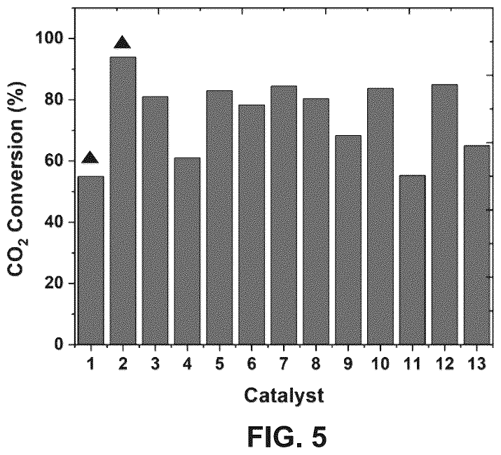

FIG. 5 illustrates a bar chart depicting carbon dioxide conversion of various catalysts during carbon dioxide methanation, according to some embodiments. FIG. 5 summarizes the CO₂ conversion values during the carbon dioxide methanation reaction for catalysts of the present disclosure and comparison catalysts. The catalysts shown in FIG. 5 correspond to the catalysts in Table 1. The Ni/CePr/GR3 catalyst showed exceptional CO₂ conversion as well as selectivity (no CO produced) compared to comparison catalysts used for carbon dioxide methanation reaction.

TABLE 1
Carbon Dioxide Conversions for Various Catalysts.

| | | Reaction | | Gas mixture | H₂:CO₂ | | | |
|---|---|---|---|---|---|---|---|---|
| | | Temperature | Flow rate | CO₂:H₂:Inert | v/v | GHSV | X_{CO2} | S_{CH4} |
| No. | Catalyst name | (C.) | (ml min⁻¹) | (mL min⁻¹) | ratio | (mLg⁻¹h⁻¹) | (%) | (%) |
| 1 | Ni/GR3 | 350 | 100 | 10:40:50 | 4 | 25000 | 55 | 93 |
| 2 | Ni/CePr/GR3 | 350 | 100 | 10:40:50 | 4 | 25000 | 94 | 99 |
| 3 | 11 wt. % Ni/N-CNTs | 360 | 200 | 20:80:100 | 4 | 60000 | 81 | 98 |
| 4 | 12Ni/CNT | 350 | 50 | 10:40:00 | 4 | 30000 | 61 | 97 |
| 5 | 12 wt. % Ni-4.5 | 350 | 50 | 10:40:00 | 4 | 30000 | 83 | 97 |
| | wt. % Ce/N-CNTs | | | | | | | |
| 6 | 20 wt. % Ni/RGO | 350 | 30 | | 4 | 36000 | 78 | 98 |
| 7 | 20 wt. % Ni-5 | 350 | 30 | | 4 | 36000 | 85 | 98 |
| | wt. % Ce/RGO | | | | | | | |
| 8 | Ni/GA | 350 | 30 | | 4 | 36000 | 80 | 94.8 |
| 9 | Ni/GO | 350 | 30 | | 4 | 36000 | 68 | 93.5 |
| 10 | Ni—SiO₂/GO-Ni-foam | 470 | | | 4 | 500 | 83 | 98 |
| 11 | Ni₁₅/rGO | 240 | | | 4 | | 55 | 100 |
| 12 | Ni/CNF-FLG | 360 | | | 4 | 30000 | 85 | 100 |
| 13 | nitrogen doped | 400 | | 18:72:10 | 4 | 18000 | 65 | 55 |
| | graphene quantum | | | | | | | |
| | dots (NGQDs) | | | | | | | |

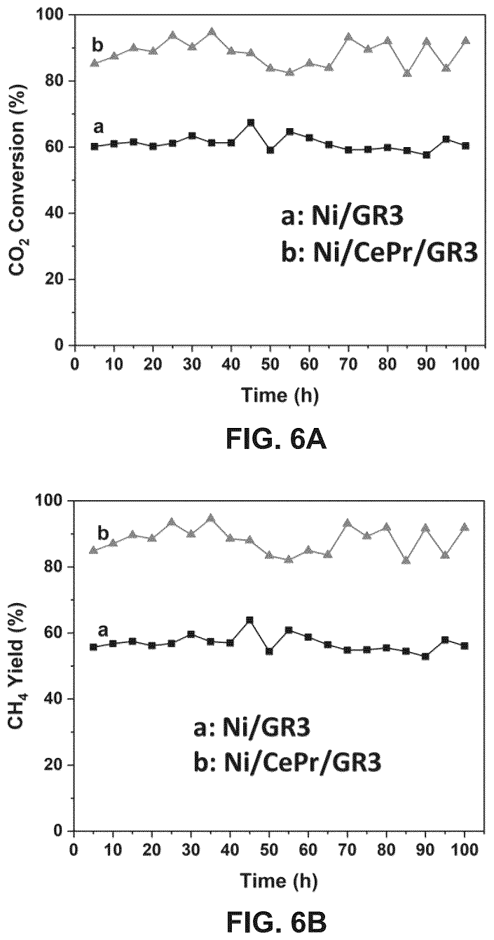

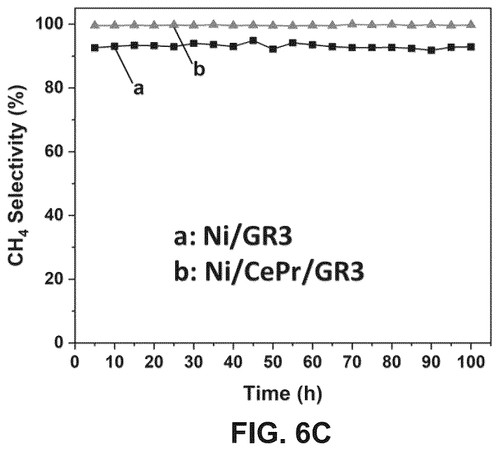

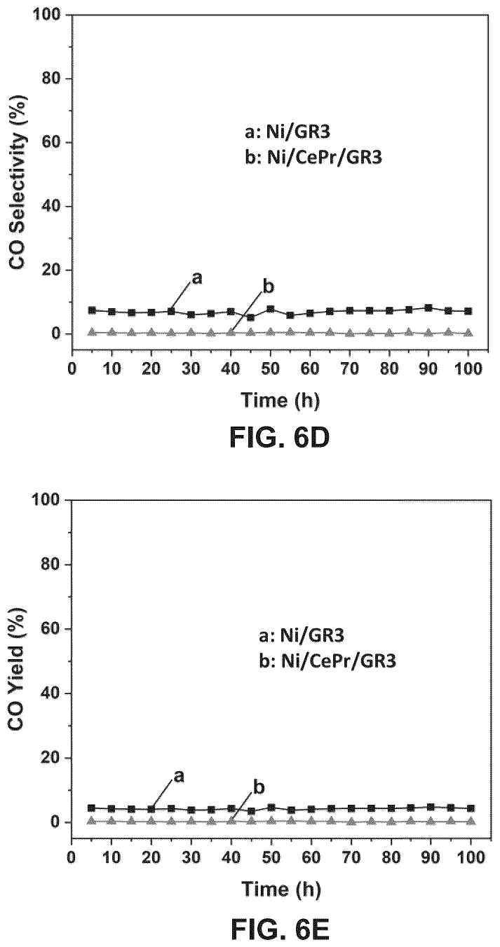

FIG. 6A illustrates stability testing (carbon dioxide conversion) for a Ni/GR3 catalyst and a Ni/CePr/GR3 catalyst, according to some embodiments. FIG. 6B illustrates stability testing (methane yield) for a Ni/GR3 catalyst and a Ni/CePr/GR3 catalyst, according to some embodiments. FIG. 6C illustrates stability testing (methane selectivity) for a Ni/GR3 catalyst and a Ni/CePr/GR3 catalyst, according to some embodiments. FIG. 6D illustrates stability testing (carbon monoxide selectivity) for a Ni/GR3 catalyst and a Ni/CePr/GR3 catalyst, according to some embodiments. FIG. 6E illustrates stability testing (carbon monoxide yield) for a Ni/GR3 catalyst and a Ni/CePr/GR3 catalyst, according to some embodiments. The reaction conditions for FIG. 6A-6E are as follows: WHSV=25,000 mL g_{cat}⁻¹ h⁻¹, 350° C., 1 bar, H₂:CO₂=4:1, and 100 h time on stream. As shown, and for example, the addition of CePr enhanced the carbon dioxide conversion and the methane yield.

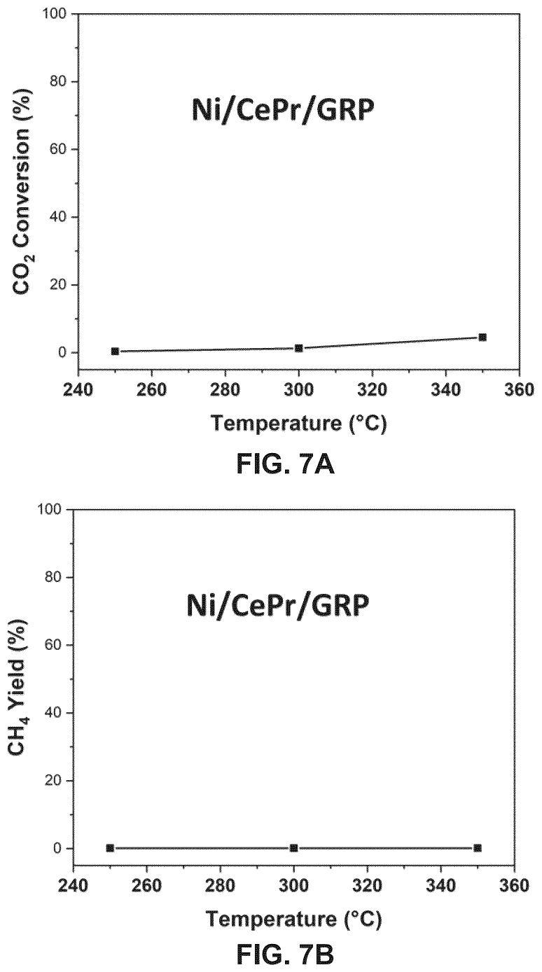

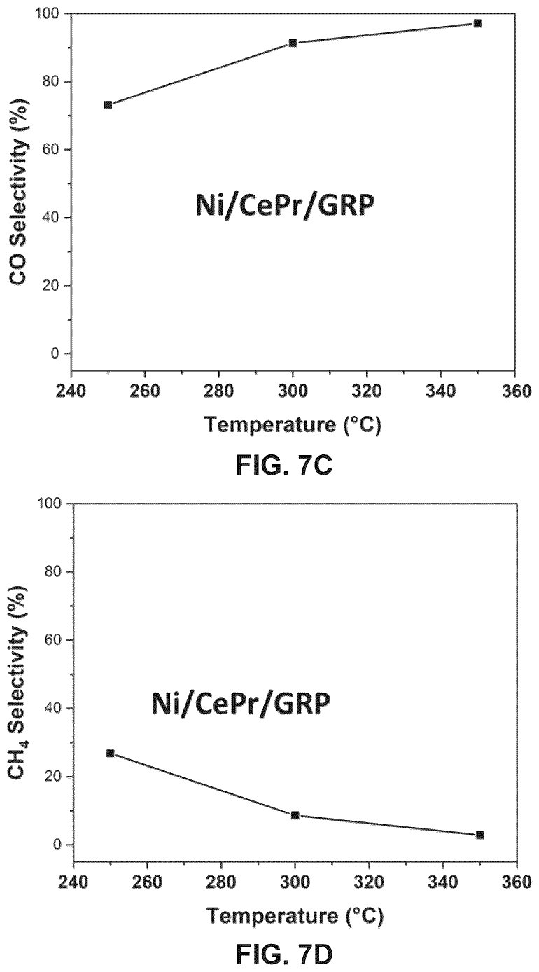

FIG. 7A illustrates stability testing (carbon dioxide conversion) for a Ni/CePr/GRP catalyst, according to some embodiments. FIG. 7B illustrates stability testing (methane yield) for a Ni/CePr/GRP catalyst, according to some embodiments. FIG. 7C illustrates stability testing (carbon monoxide selectivity) for a Ni/CePr/GRP catalyst, according to some embodiments. FIG. 7D illustrates stability testing (methane selectivity) for a Ni/CePr/GRP catalyst, according to some embodiments. The reaction conditions for FIG. 7A-7D are as follows: WHSV=25,000 mL gcat⁻¹ h⁻¹, 350° C., 1 bar, H₂:CO₂=4:1, and 100 h time on stream. The ability of graphene to donate electrons can enhance the metal-support interaction which prevents sintering and stabilizes the catalyst and thus prevents or reduces its deactivation. This is shown in FIGS. 6A-6E and FIG. 7A-7D, where the stability of the catalyst with time is shown for 100 hr time on stream. The GRP-based catalyst was produced using the pyrolysis-based graphene-like material. For example, lower conversion values of CO₂ were recorded compared to the Ni/CePr/GR3 catalyst. In one example, this can be attributed to the poor graphene structure in the pyrolysis-formed graphene material (less resemblance to graphene) and the higher level of impurities in the pyrolysis-formed graphene (e.g. Fe, Si) compared to the Ni/CePr/GR3 catalyst.

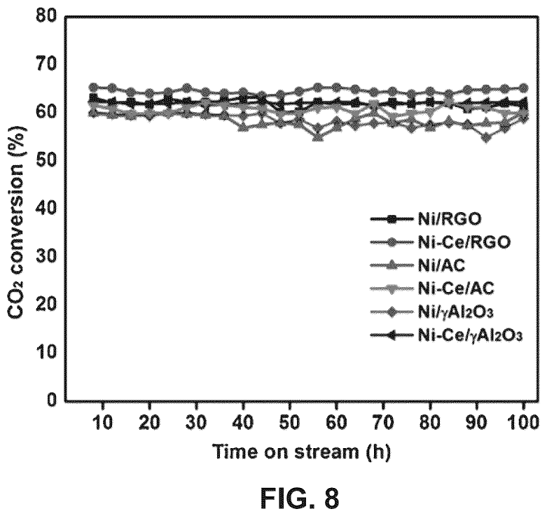

FIG. 8 illustrates a comparative example of carbon dioxide conversion of various catalysts, according to some embodiments. The reaction conditions are as follows: GHSV=36,000 h⁻¹, 450° C., 1 atm, and H₂:CO₂=4:1. As shown in FIG. 6A, the carbon dioxide conversion percentage for the Ni/CePr/GR3 catalyst is superior compared to the comparison catalysts in FIG. 8.

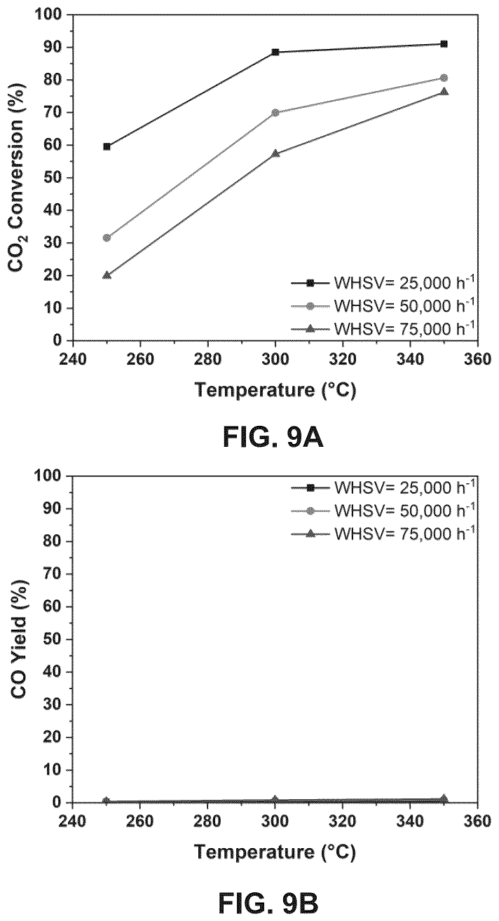

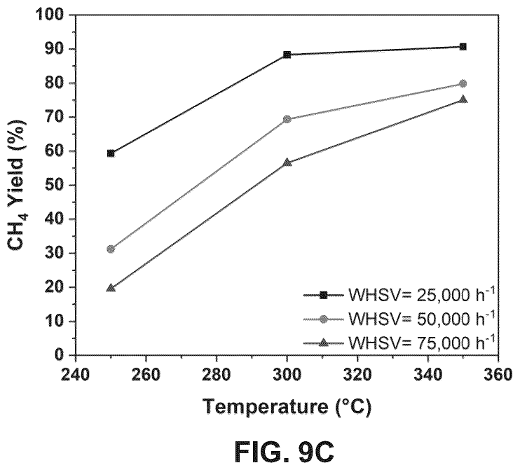

FIG. 9A illustrates the effect of temperature and space velocity on carbon dioxide methanation performance (carbon dioxide conversion) for the Ni/CePr/GR3 catalyst, according to some embodiments. FIG. 9B illustrates the effect of temperature and space velocity on carbon dioxide methanation performance (carbon monoxide yield) for the Ni/CePr/GR3 catalyst, according to some embodiments. FIG. 9C illustrates the effect of temperature and space velocity on carbon dioxide methanation performance (methane yield) for the Ni/CePr/GR3 catalyst, according to some embodiments. In this study, 3 different space velocities were tested: 25,000 h⁻¹, 50,000 h⁻¹ and 75,000 h⁻¹. The catalyst performs exceptionally well and stable even at the highest space velocity studied, confirming excellent overall performance for the methanation reaction.

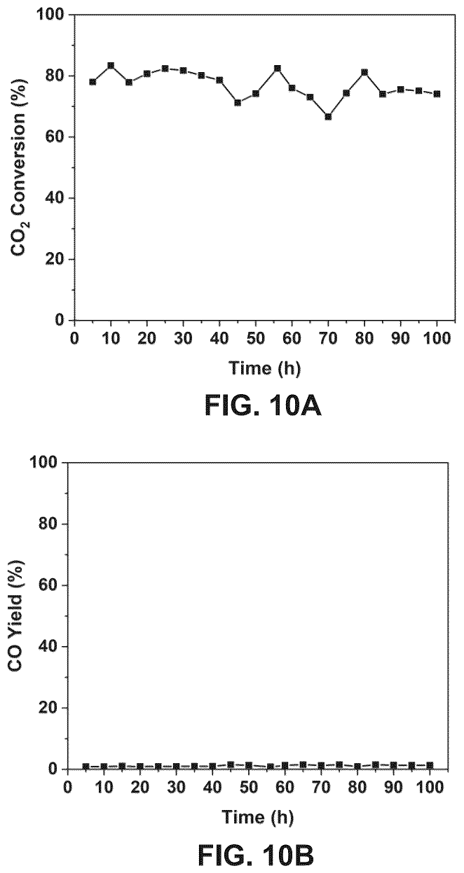

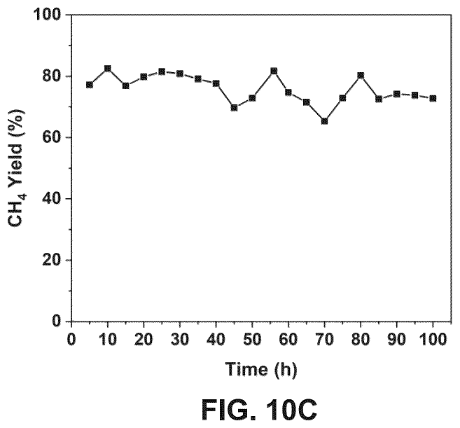

FIG. 10A illustrates the effect of time on stream on the carbon dioxide methanation performance (carbon dioxide conversion) of the Ni/CePr/GR3 catalyst, according to some embodiments. FIG. 10B illustrates the effect of time on stream on the carbon dioxide methanation performance (carbon monoxide yield) of the Ni/CePr/GR3 catalyst, according to some embodiments. FIG. 10C illustrates the effect of time on stream on the carbon dioxide methanation performance (methane yield) of the Ni/CePr/GR3 catalyst, according to some embodiments. The tested reaction conditions are as follows: WHSV=75,000 mL gcat⁻¹ h⁻¹, 350° C., 1 bar, H₂:CO₂=4:1, and 100 h time on stream. FIG. 10A-FIG. 10C showcase the excellent stability of the Ni/CePr/GR3 catalyst, even for 100 hours on stream.

### Example 2—Sulfur Addition to Graphene-Containing Support

Usually, the industrial gas feeds of CO₂ contain sulfur impurities. Conventionally, nickel-based catalysts are greatly sensitive to sulfur-containing impurities in the feedstock. Even very low levels of sulfur can greatly deactivate Ni catalysts by forming nickel sulfide phases such as NiS and Ni₃S₂. One sulfur atom can poison ten or more Ni active sites due to its geometric blocking and electronic effects.

To prepare the catalysts supports, Pr(NO₃)₃ was added into an aqueous Ce(NO₃)₃ solution (0.122 M) at a Ce³⁺:Pr³⁺ ratio of 4:1 (80 wt. %: 20 wt. %). Then, the solution was stirred at 65° C. for 21 h 15 min. Subsequently, a 2.5 ml of 25% ammonia solution was added to promote precipitation and stirred for 2 h or until dry. The resulting precipitate was transferred into a crucible for calcination at 350° C. for 4 h in static air. Supported Ni catalysts were prepared by wet impregnation. In a typical procedure, the support material was added to an aqueous solution containing a 10 wt % of Ni(NO₃)₂ under rigorous stirring at 95° C. (Temperature of hot plate) overnight until dry. The catalysts were obtained by calcining at 350° C. for 4 h in air.

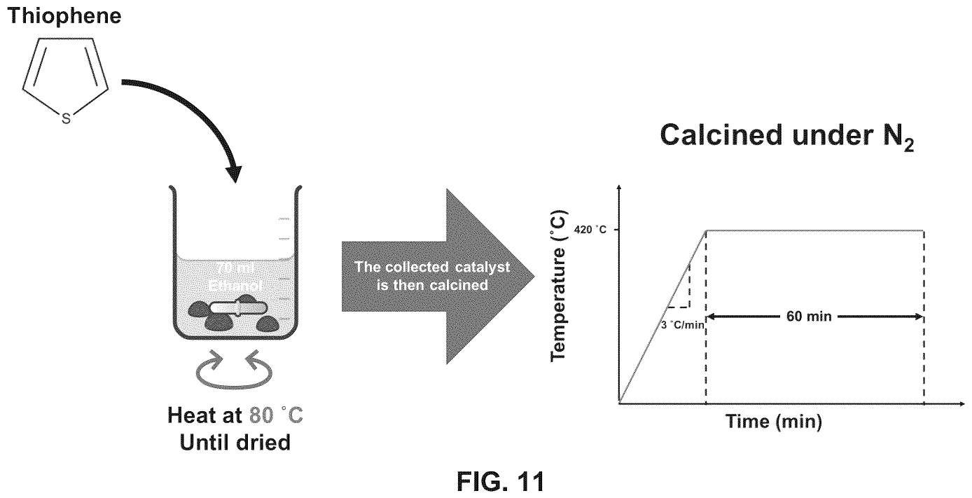

FIG. 11 illustrates sulfur addition to the Ni/CePr/GR3 catalyst, according to some embodiments. To study the effect of sulfur poisoning, the fresh catalyst was placed in 70 ml of ethanol and poisoned with 1 wt. % S using thiophene. This mixture was heating at 80° C. (temperature of hotplate) until the mixture was dry. The collected powder was the calcined under N₂ for 1 h at 420° C. The 1 wt. % loading was performed to simulate the sulfur content of catalysts in feed streams including H₂S and/or SO₂ feeds.

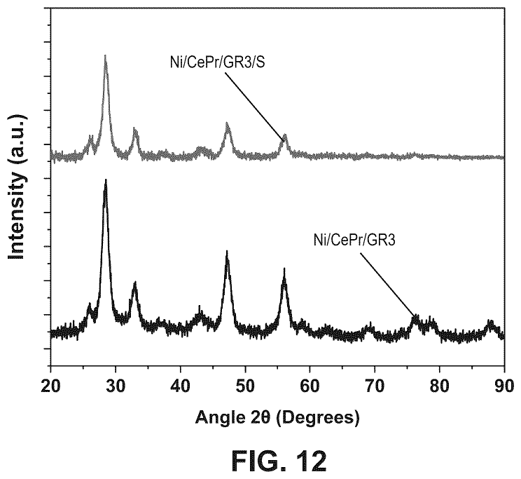

FIG. 12 illustrates x-ray diffraction (XRD) patterns of a Ni/CePr/GR3 and a Ni/CePr/GR3/S catalyst with 1 wt. % sulfur, according to some embodiments. Both fresh and poisoned with lwt % of S shows the diffraction peaks of cubic fluorite structure of CeO₂ at around 28.5, 33.1, 47.5 and 56°, which correspond to reflections due to the (111), (200), (220) and (311) planes, respectively. Additionally, the peaks at around 25.5° and 42.92° indicate the distance between graphene layers and (100) diffraction plane, respectively. The diffraction peaks at 37.2°, 43.2°, and 62.8° appear in the case of the 10 wt. % Ni impregnated carriers, which correspond to (111), (200), and (220) diffraction planes of face-centered cubic phase NiO.

The catalytic performance toward carbon dioxide methanation was tested using a continuous flow fixed-bed stainless steel reactor. 0.24 g of the fresh catalyst was loaded in the reactor and reduced for 3 h using 50% H₂/He flow. Then the catalytic activity was evaluated as a function of temperature (T=250° C., 300° C., 350° C.) with a total flow rate of 100 mL min⁻¹ (10 mL min⁻¹ CO₂, 40 mL min⁻¹ H₂ and 50 mL min-1 He) and weight hourly space velocity (WHSV) of 25,000 mL gcat⁻¹ h⁻¹ and the temperature was maintained for 1 h. The effluent gases were continuously monitored using a GC-MS. For example, CO was detected as the only reaction by-product besides CH₄. CH₄ selectivity, CO selectivity and CH₄ yield were calculated based on the following Eqs. (2)-(5). The stability tests (time-on-stream) were conducted for 100 h at 350° C. (high activity was achieved in this temperature) under 10 mL min⁻¹ CO, 40 mL min⁻¹ H₂ and 50 mL min⁻¹ He.

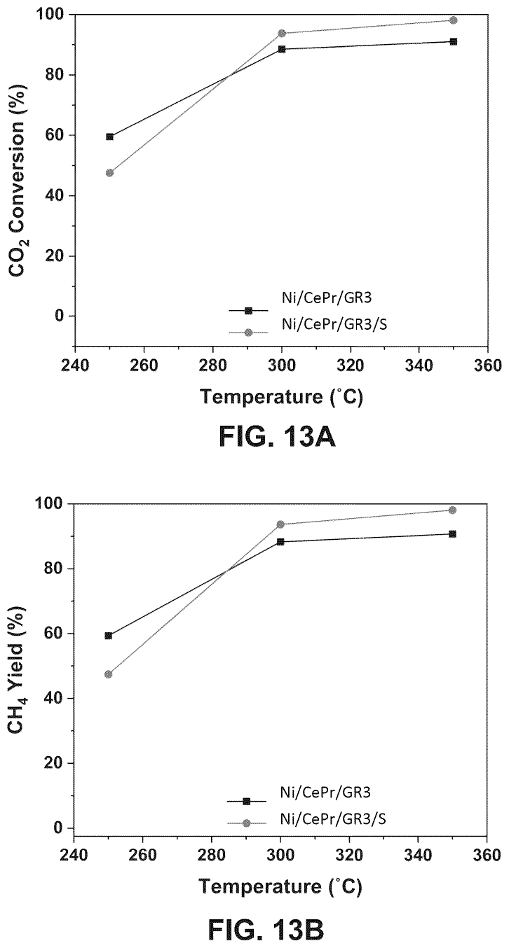

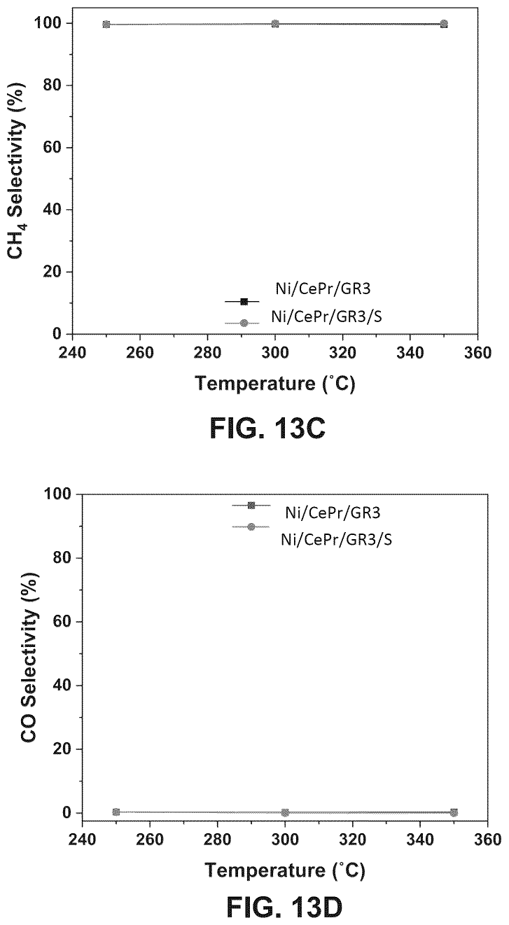

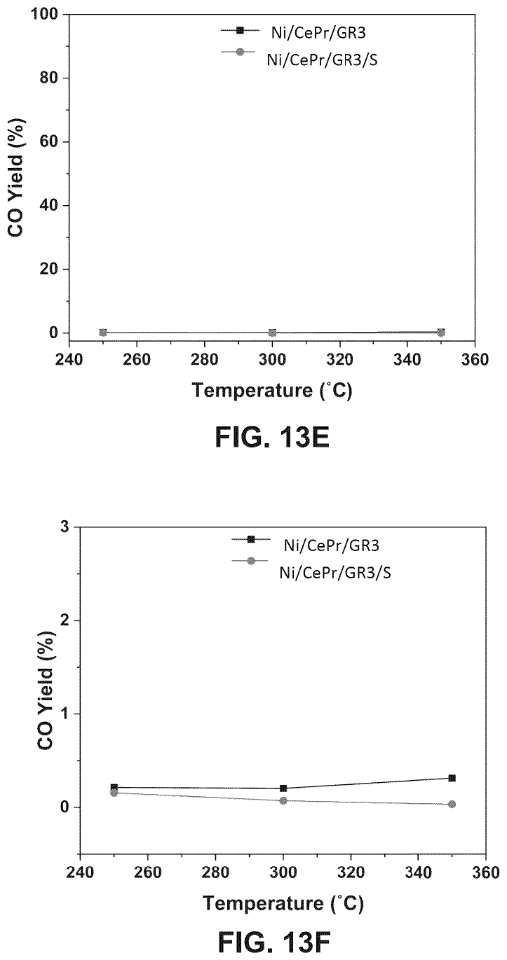

FIG. 13A illustrates performance (carbon dioxide conversion) of the Ni-supported catalysts (Ni/CePr/GR3 and Ni/CePr/GR3/S) as a function of reaction temperature, according to some embodiments. FIG. 13B illustrates performance (methane yield) of the Ni-supported catalysts (Ni/CePr/GR3 and Ni/CePr/GR3/S) as a function of reaction temperature, according to some embodiments. FIG. 13C illustrates performance (methane selectivity) of the Ni-supported catalysts (Ni/CePr/GR3 and Ni/CePr/GR3/S) as a function of reaction temperature, according to some embodiments. FIG. 13D illustrates performance (carbon monoxide selectivity) of the Ni-supported catalysts (Ni/CePr/GR3 and Ni/CePr/GR3/S) as a function of reaction temperature, according to some embodiments. FIG. 13E illustrates performance (carbon monoxide yield) of the Ni-supported catalysts (Ni/CePr/GR3 and Ni/CePr/GR3/S) as a function of reaction temperature, according to some embodiments. FIG. 13F illustrates performance (magnified carbon monoxide yield) of the Ni-supported catalysts (Ni/CePr/GR3 and Ni/CePr/GR3/S) as a function of reaction temperature, according to some embodiments. Reaction conditions: WHSV=25,000 mL g⁻¹ h⁻¹; H₂/CO₂ molar ratio=4; P=1 atm; T=250° C., 300° C., and 350° C.

This shows the CO₂ conversion and CH₄ selectivity of Ni/CePr/GR3 fresh catalyst compared with the poisoned catalyst at different temperatures. It is evident that with increasing reaction temperature from 250° C. to 350° C., a significant improvement in CO₂ conversion and CH₄ selectivity is shown due to the kinetics of the reaction. For example, the poisoned catalyst Ni/CePr/GR3/S outperformed Ni/CePr/GR3 in terms of carbon dioxide conversion at the process temperatures. The poisoned catalyst even decreased the yield of CO indicating almost no side reactions are occurring simultaneously.

### Example 3—Graphite-Containing Support

To prepare the catalysts supports, Pr(NO₃)₃ was added into an aqueous Ce(NO₃)₃ solution (0.122 M) at a Ce³⁺:Pr³⁺ ratio of 4:1 (80 wt. %: 20 wt. %). Then, the solution was stirred at 65° C. for 21 h 15 min. Subsequently, a 2.5 ml of 25% ammonia solution was added to promote precipitation and stirred for 2 h or until dry. The resulting precipitate was transferred into a crucible for calcination at 350° C. for 4 h in static air. Supported Ni catalysts were prepared by wet impregnation. In a typical procedure, the support material (graphite—GT) was added to an aqueous solution containing a 10 wt % of Ni(NO₃)₂ under rigorous stirring at 95° C. (Temperature of hot plate) overnight until dry. The catalysts were obtained by calcining at 350° C. for 4 h in air.

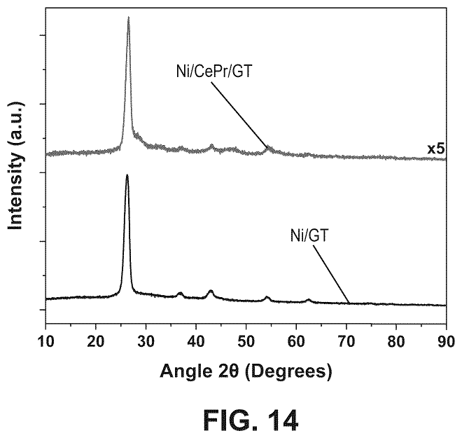

FIG. 14 illustrates x-ray diffraction (XRD) patterns for Ni/CePr/GT and Ni/GT, according to some embodiments. The collected X-ray Diffraction (XRD) of Ni/GT and Ni/CePr/GT shows the diffraction peaks of hexagonal graphite at around 26.4°, 54.5°, 83.2°, and 86.9° which correspond to (002), (101), (004), and (006), respectively. After the addition of CePrO metal oxide promoter, the Ni/CePr/GT shows a cubic fluorite structure of CeO₂ with diffraction peaks at around 28.5, 33.1, 47.5 and 56°, which correspond to reflections due to the (111), (200), (220) and (311) planes, respectively.

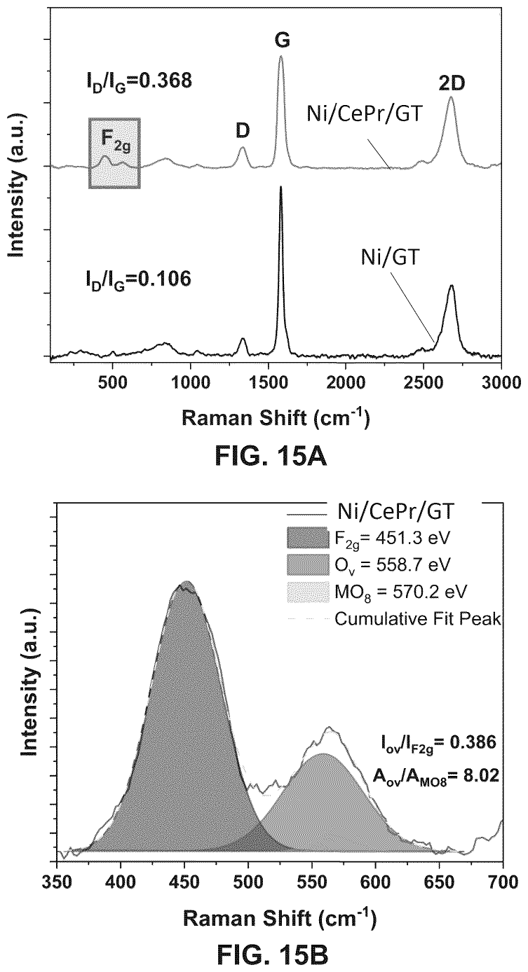

FIG. 15A illustrates Raman spectra for Ni/CePr/GT and Ni/GT, according to some embodiments. FIG. 15B illustrates deconvoluted Raman spectra for Ni/CePr/GT and Ni/GT, according to some embodiments. The D, G, and 2D or G′ disorder bands that appear at around 1320-1350 cm⁻¹, 1570-1585 cm⁻¹, and 2640-2680 cm⁻¹ can correspond to the disorder-induced characteristic from sp³-bonded carbon and defects (D band) and n-plane bond stretching vibration of the sp²-hybridized carbon (G and 2D), respectively. These bands were visible in the Raman spectra of GT samples.

The ID/IG ratio can be used to quantify the disorder and defects among different graphene-based materials, which can change surface properties and adsorption capability. The higher the ID/IG ratios, the more disorder. It was observed that adding CePrO promoter increases the ID/IG from 0.106 to 0.368 (3.5 higher). The Raman spectra of the CePrO promoted catalyst exhibit the peaks of ceria-based metal oxides (F2 g˜461 cm−1 and Ov/MO8˜590 cm⁻¹), as confirmed by XRD results. This shift is correlated to disturbances in symmetry of the oxygen sublattice (Ce-08) caused by thermal or dimensional effects (doping). The Iov/IF2 g intensity ratio was used as descriptor for oxygen vacancy (O_{V}). The higher the Iov/IF2 g ratio the higher the oxygen vacant sites formed. In the case of Ni/CePrO-GR3 catalyst (Iov/IF2 g=0.22) has a Iov/IF2 g ratio c.a.1.75 times lower than Ni/CePr/GT. For example, this can explain the enhanced catalytic performance of Ni/CePr/GT catalyst (Iov/IF2 g=0.386).

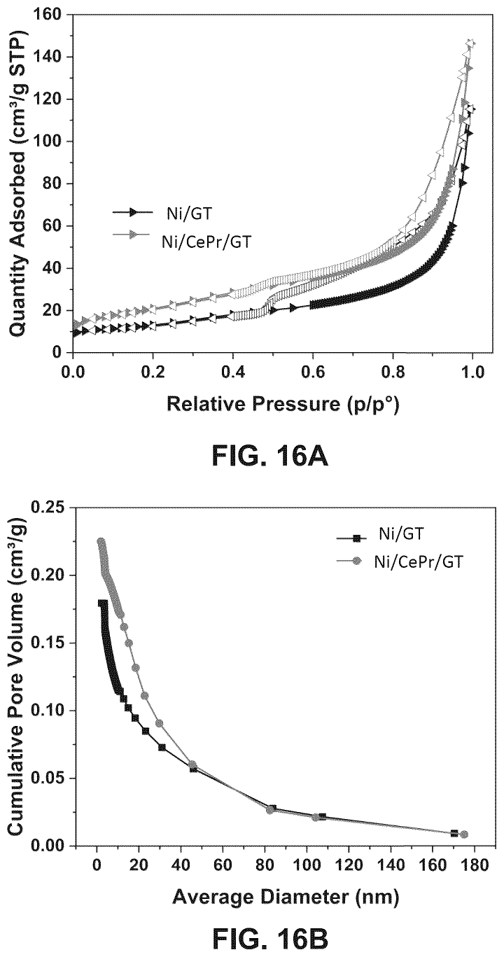

FIG. 16A illustrates N₂ adsorption-desorption isotherms for Ni/CePr/GT and Ni/GT, according to some embodiments. FIG. 16B illustrates the cumulative pore volume for Ni/CePr/GT and Ni/GT, according to some embodiments. All the isotherms are type-IV with type-H3 hysteresis, indicating N₂ condensation in meso-pores during adsorption. Interestingly, the 10 wt. % impregnation and the addition of CePrO lead to an increase in specific surface area (SSA). The metal-oxides added to GR3 and GT act as stabilizers that prevent the agglomeration and restacking of graphene sheets. Whereas, the pore size slightly decreased with the addition of metal-oxides, as shown Table 2. For example, while the catalysts supported on GR3 have higher SSA than GT carriers, the Ni/CePr/GT catalysts showed similar catalytic activity at 350° C. and slightly higher CO₂ conversion at 100 h TOS than Ni/CePrO-GR3 catalyst.

TABLE 2
BET Surface Area, Pore Volume, and Pore
Size of Catalysts and Catalyst Supports.

| | | S_{BET} | Vₚ | Dₚ |
|---|---|---|---|---|
| No. | Sample | (m²/g) | (cm³/g) | (nm) |
| 1 | GR3 | 86.47 | 0.037 | 18.9 |
| 2 | GRP | 93.52 | 0.463 | 19.8 |
| 3 | CePrO-GR3 | 114.69 | 0.393 | 13.7 |
| 4 | Ni/GR3 | 130.4 | 0.589 | 18.1 |
| 5 | Ni/CePrO-GR3 | 108.7 | 0.422 | 15.5 |
| 6 | Ni/CePrO-GRP | 123.37 | 0.334 | 10.8 |
| 7 | Ni/GT | 47.6 | 0.174 | 14.6 |
| 8 | Ni/CePrO-GT | 76.6 | 0.222 | 11.6 |

The catalytic performance of the catalysts toward carbon dioxide methanation was tested using a continuous flow fixed-bed stainless steel reactor. 0.144 g of the fresh catalyst was loaded in the reactor and reduced for 3 h using 50% H₂/He flow. Then the catalytic activity was evaluated as a function of temperature (T=250° C., 300° C., 350° C.) with a total flow rate of 60 mL min⁻¹ (6 mL min⁻¹ CO₂, 24 mL min⁻¹ H₂ and 30 mL min⁻¹ He) and weight hourly space velocity (WHSV) of 25,000 mL g_{cat}⁻¹ h⁻¹ and the temperature was maintained for 1 h. The effluent gases were continuously monitored using a GC-MS. For example, CO was detected as the only reaction by-product besides CH₄. CH₄ selectivity, CO selectivity and CH₄ yield were calculated based on the following Eqs. (2)-(5). The stability tests (time-on-stream) were conducted for 100 h at 350° C. (chosen as high activity was achieved in this temperature) under 10 mL min⁻¹ CO, 40 mL min⁻¹ H₂ and 50 mL min⁻¹ He.

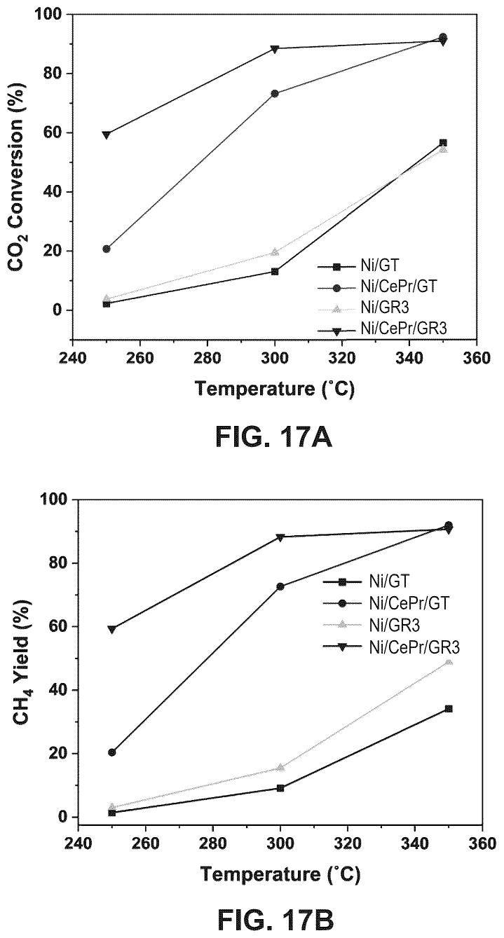

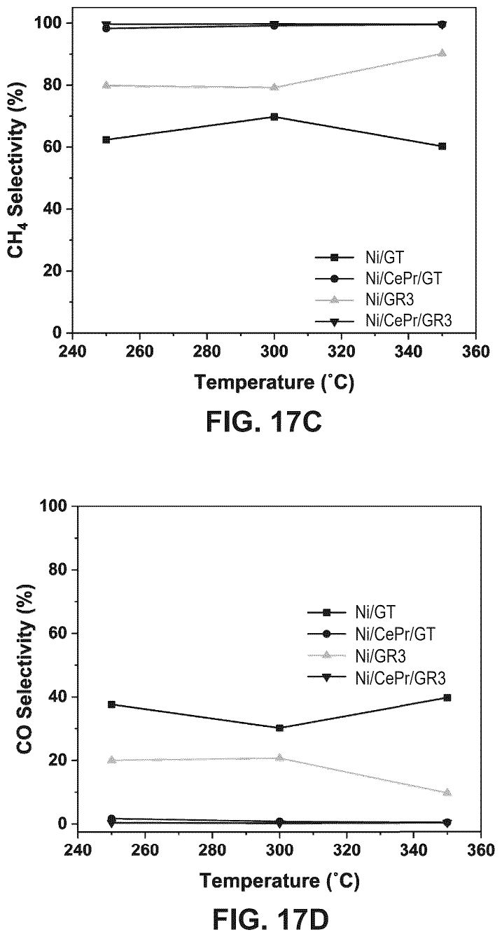

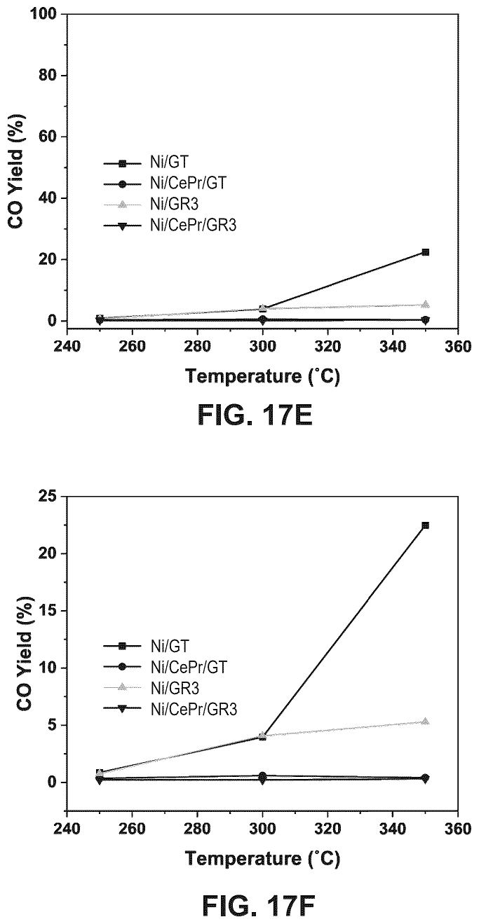

FIG. 17A illustrates performance (carbon dioxide conversion) of the Ni-supported catalysts (Ni/GR3, Ni/CePr/GR3, Ni/GT, and Ni/CePr/GT) as a function of reaction temperature, according to some embodiments. FIG. 17B illustrates performance (methane yield) of the Ni-supported catalysts (Ni/GR3, Ni/CePr/GR3, Ni/GT, and Ni/CePr/GT) as a function of reaction temperature, according to some embodiments. FIG. 17C illustrates performance (methane selectivity) of the Ni-supported catalysts (Ni/GR3, Ni/CePr/GR3, Ni/GT, and Ni/CePr/GT) as a function of reaction temperature, according to some embodiments. FIG. 17D illustrates performance (carbon monoxide selectivity) of the Ni-supported catalysts (Ni/GR3, Ni/CePr/GR3, Ni/GT, and Ni/CePr/GT) as a function of reaction temperature, according to some embodiments.

FIG. 17E illustrates performance (carbon monoxide yield) of the Ni-supported catalysts (Ni/GR3, Ni/CePr/GR3, Ni/GT, and Ni/CePr/GT) as a function of reaction temperature, according to some embodiments. FIG. 17F illustrates performance (magnified carbon monoxide yield) of the Ni-supported catalysts (Ni/GR3, Ni/CePr/GR3, Ni/GT, and Ni/CePr/GT) as a function of reaction temperature, according to some embodiments. The reaction conditions are as follows: WHSV=25,000 mL g⁻¹ h⁻¹; H₂/CO₂ molar ratio=4; P=1 atm; T=250° C., 300° C., and 350° C. It is shown that with increasing reaction temperature from 250° C. to 350° C., a significant improvement in CO₂ conversion and CH₄ selectivity is shown. In one example, the Ni/CePr/GT outperformed Ni/CePr/GR3 in terms of CO₂ conversion and CO selectivity.

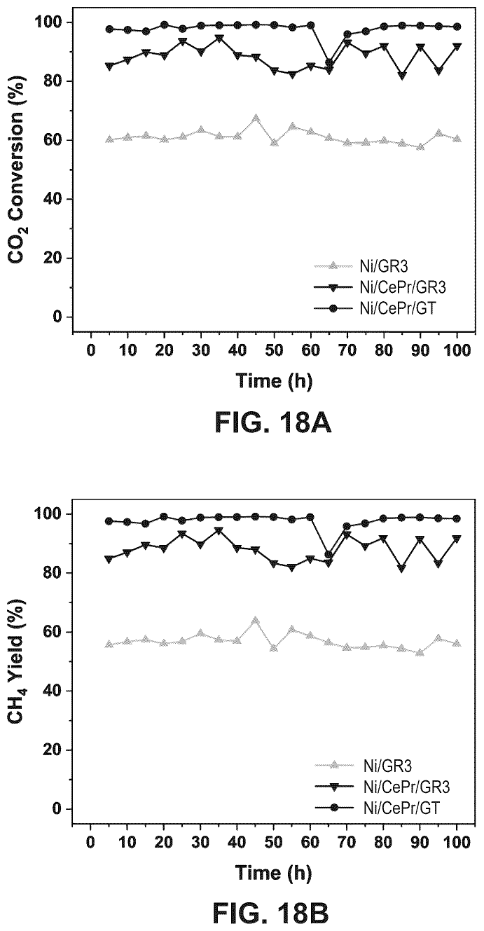

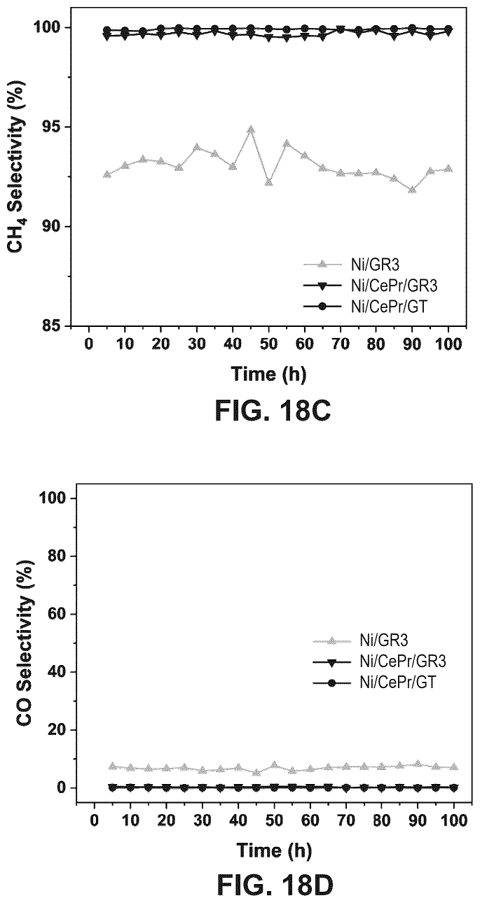

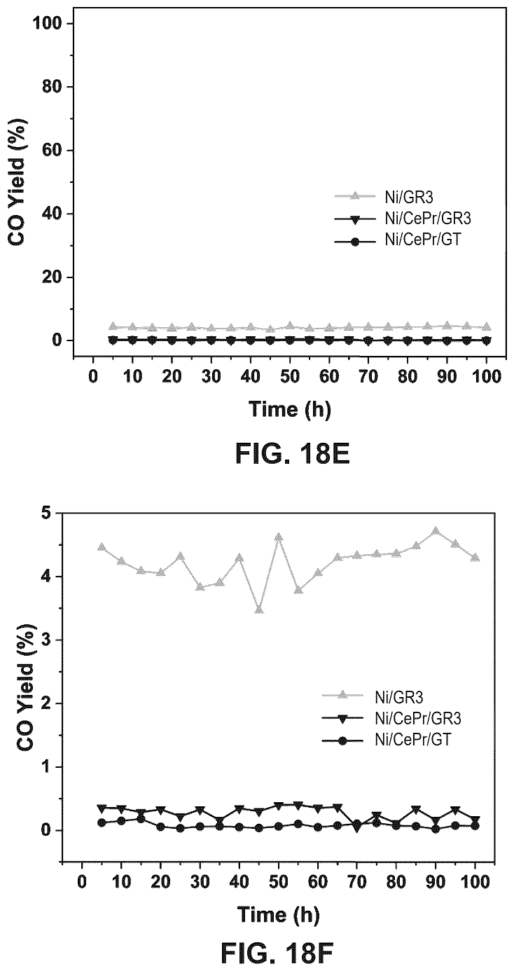

FIG. 18A illustrates stability testing (carbon dioxide conversion) of the Ni-supported catalysts (Ni/GR3, Ni/CePr/GR3, and Ni/CePr/GT) as a function of reaction temperature, according to some embodiments. FIG. 18B illustrates stability testing (methane yield) of the Ni-supported catalysts (Ni/GR3, Ni/CePr/GR3, and Ni/CePr/GT) as a function of reaction temperature, according to some embodiments. FIG. 18C illustrates stability testing (methane selectivity) of the Ni-supported catalysts (Ni/GR3, Ni/CePr/GR3, and Ni/CePr/GT) as a function of reaction temperature, according to some embodiments. FIG. 18D illustrates stability testing (carbon monoxide selectivity) of the Ni-supported catalysts (Ni/GR3, Ni/CePr/GR3, and Ni/CePr/GT) as a function of reaction temperature, according to some embodiments.

FIG. 18E illustrates stability testing (carbon monoxide yield) of the Ni-supported catalysts (Ni/GR3, Ni/CePr/GR3, and Ni/CePr/GT) as a function of reaction temperature, according to some embodiments. FIG. 18F illustrates stability testing (carbon monoxide yield) of the Ni-supported catalysts (Ni/GR3, Ni/CePr/GR3, and Ni/CePr/GT) as a function of reaction temperature, according to some embodiments. The reaction conditions are as follows: T=350° C., WHSV=25,000 mL g⁻¹ h⁻¹; H₂/CO₂ molar ratio=4; P=1 atm; TOS=100 h. Using the GT support further improved the metal-support interaction when comparted to the GR3 support. This prevented sintering and stabilized the catalyst.

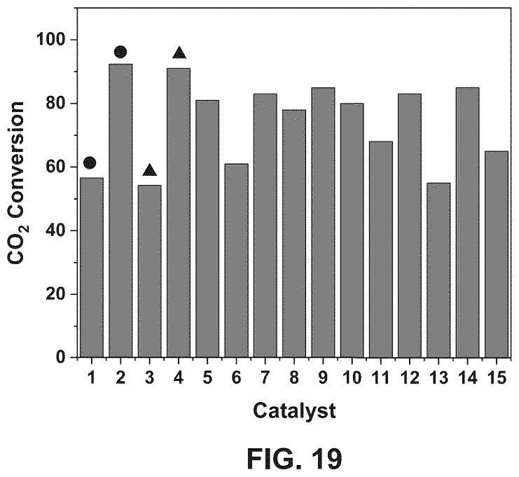

FIG. 19 illustrates a bar chart depicting carbon dioxide conversion of various catalysts during carbon dioxide methanation, according to some embodiments. The catalysts shown in FIG. 19 correspond to Table 3. In one example, the Ni/CePr/GT costs 46.25% less to synthesize compared to Ni/CePr/GR3. In another example, the yield of Ni/CePr/GT is 50.77% higher than Ni/CePr/GR3. The Ni/CePr/GT catalyst also outperformed the comparison catalysts described in Table 3.

TABLE 3
Carbon Dioxide Conversions for Various Catalysts.

| | | Reaction | | Gas mixture | H₂:CO₂ | | | |
|---|---|---|---|---|---|---|---|---|
| | | Temperature | Flow rate | CO₂:H₂:Inert | v/v | GHSV | X_{CO2} | S_{CH4} |
| No. | Catalyst | (° C.) | (mL min⁻¹) | (mL min⁻¹) | ratio | (mLg⁻¹h⁻¹) | (%) | (%) |
| 1 | 10% Ni/GRT | 350 | 60 | 10:40:50 | 4 | 25000 | 57 | 60 |
| 2 | 10% Ni/20% CePrO-GT | 350 | 60 | 10:40:50 | 4 | 25000 | 92 | 99.6 |
| 3 | 10% Ni/GR3 | 350 | 100 | 10:40:50 | 4 | 25000 | 55 | 93 |
| 4 | 10% Ni/20% (Ce—Pr)GR3 | 350 | 100 | 10:40:50 | 4 | 25000 | 91.02 | 99.66 |
| 5 | 11 wt. % Ni/N-CNTs | 360 | 200 | 20:80:100 | 4 | 60000 | 81 | 98 |
| 6 | 12Ni/CNT | 350 | 50 | 10:40:00 | 4 | 30000 | 61 | 97 |
| 7 | 12 wt. % Ni-4.5 | 350 | 50 | 10:40:00 | 4 | 30000 | 83 | 97 |
| | wt. % Ce/N-CNTs | | | | | | | |
| 8 | 20 wt. % Ni/RGO | 350 | 30 | | 4 | 36000 | 78 | 98 |
| 9 | 20 wt. % Ni-5 | 350 | 30 | | 4 | 36000 | 85 | 98 |
| | wt. % Ce/RGO | | | | | | | |
| 10 | Ni/GA | 350 | 30 | | 4 | 36000 | 80 | 94.8 |
| 11 | Ni/GO | 350 | 30 | | 4 | 36000 | 68 | 93.5 |
| 12 | Ni—SiO₂/GO-Ni-foam | 470 | | | 4 | 500 | 83 | 98 |
| 13 | Ni₁₅/rGO | 240 | | | 4 | | 55 | 100 |
| 14 | Ni/CNF-FLG | 360 | | | 4 | 30000 | 85 | 100 |
| 15 | nitrogen doped | 400 | | 18:72:10 | 4 | 18000 | 65 | 55 |
| | graphene quantum | | | | | | | |
| | dots (NGQDs) | | | | | | | |

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A catalyst, comprising:
a catalytically active material; and
a support including:
a carbon-containing material including at least one of graphene and graphite, wherein the carbon-containing material includes less than 5 wt. % oxygen based on elemental analysis of the carbon-containing material; and
at least one of cerium oxide and praseodymium oxide.

2. The catalyst of claim 1, wherein the support includes cerium oxide and praseodymium oxide.

3. The catalyst of claim 2, wherein a weight ratio of cerium to praseodymium ranges from about 2:1 to about 6:1, based on elemental analysis.

4. The catalyst of claim 1, wherein the catalytically active material includes nickel.

5. The catalyst of claim 1, wherein the support includes graphene, and wherein a weight percentage of carbon in the graphene is greater than 98 wt. %.

6. The catalyst of claim 1, wherein the support includes graphene, and wherein the graphene includes plasma-formed graphene.

7. The catalyst of claim 1, wherein the support includes graphite.

8. The catalyst of claim 7, wherein the graphite includes laser-expanded graphite.

9. The catalyst of claim 1, wherein a weight percentage of nickel in the catalyst ranges from about 5 wt. % to about 30 wt. %.

10. The catalyst of claim 1, wherein the catalyst exhibits a BET surface area of greater than about 70 m²/g, and wherein the support exhibits an average pore size ranging from about 8 nm to about 20 nm.

11. The catalyst of claim 1, further comprising sulfur in contact with at least a portion of the support.

12. A catalyst support, comprising:
graphite including less than 5 wt. % oxygen based on elemental analysis of the graphite; and
a cerium praseodymium mixed oxide, wherein a weight ratio of cerium to praseodymium ranges from about 3:1 to about 5:1.

13. The catalyst support of claim 12, wherein the catalyst support exhibits an average pore size ranging from about 8 nm to about 22 nm.

14. The catalyst support of claim 12, wherein the graphite includes laser-expanded graphite.

15. The catalyst support of claim 12, wherein a weight percentage of carbon in the graphite is greater than 95 wt. %.

16. A process for methanation, comprising:
introduce a feed stream to a catalyst at a process temperature sufficient to form methane, wherein the feed stream includes hydrogen;
wherein the catalyst includes nickel and a support including (1) at least one of graphene and graphite; and (2) at least one of cerium oxide and praseodymium oxide.

17. The process of claim 16, wherein the support includes cerium oxide and praseodymium oxide.

18. The process of claim 17, wherein a weight ratio of cerium to praseodymium ranges from about 2:1 to about 6:1, based on elemental analysis.

19. The process of claim 16, wherein the support includes graphite.

20. The process of claim 16, wherein the process temperature is greater than 300° C., and wherein the feed stream includes carbon dioxide.
